(19) United States
(12) Patent Application Publication
(10) Pub. No.: US 2021/0115370 A1
(43) Pub. Date: Apr. 22, 2021

(54) DISPOSABLE BIOREACTOR AND USE THEREOF

(71) Applicant: IRUBIS GMBH, Munich (DE)

(72) Inventors: Lorenz SYKORA, München (DE); Alexander GEISSLER, München (DE); Anja MÜLLER, Munich (DE)

(73) Assignee: IRUBIS GMBH, Munich (DE)

(21) Appl. No.: 17/253,080

(22) PCT Filed: Jun. 25, 2019
(86) PCT No.: PCT/EP2019/066831

§ 371 Date: Dec. 16, 2020

(30) Foreign Application Priority Data
Jun. 26, 2018 (DE) 10 2018 210 370.4

(51) Int. Cl. C12M 1/00; C12M 1/12; C12M 1/34

(52) U.S. Cl. CPC C12M 23/28; C12M 31/00; C12M 41/30; C12M 41/26; C12M 37/00

(57) ABSTRACT

The present invention relates to disposable bioreactors having at least one permanently integrated disposable ATR element. These bioreactors are suitable for cultivating microorganisms or cell cultures, or for cell-free gene expression, wherein liquid samples are examined in the bioreactor by means of infrared spectroscopy.

The present invention concerns single-use bioreactors with at least one permanently integrated single-use ATR element. These bioreactors are suitable for the cultivation of microorganisms, cell cultures or for cell-free gene expression. The examination of the sample liquids in the bioreactor is carried out by means of infrared spectroscopy.

Bioreactors are containers in which different organisms are cultivated under controlled and monitored conditions, such as mammalian cells which are cultivated for drug manufacturing. The container can be fabricated from materials like plastic, glass, or metal.

If bioreactors are used several times, they have to be laboriously cleaned to be sterile. Residues in the bioreactor lead to falsification and contamination of further biotechnological processes. The use of single-use bioreactors is therefore more reliable and less prone to errors.

For monitoring the biotechnical process, the sample can be measured online (in-situ). For many measurement parameters, however, a sample must be taken from the bioreactor to be analyzed with a separate measurement instrument. This leads to measurements only being taken at the time of sampling, with no continuous measurement possible. In the case of bioreactors with a very small volume, sampling can lead to a noticeable reduction in the medium, as well as increased risk of contamination.

Infrared spectroscopy is an established analytical method for the qualitative and quantitative analysis of analytes, whereby a spectrum will be obtained that is characteristic of the chemical composition of the sample.

There are different measuring principles in infrared spectroscopy. ATR infrared spectroscopy is one of the most important methods. It is based on the principle of attenuated total reflection (ATR). The analyte is directly applied to the ATR element and the light is coupled to the ATR element from the bottom. At the interface between the ATR element and the analyte, some of the light interacts with the sample and is absorbed by it.

U.S. Pat. No. 9,267,100B2 discloses a composite sensor assembly for bioreactors. These sensors are not integrated directly in the bioreactor, but in an assembled unit. This assembled unit is glued into the bioreactor.

Single-use connections for bioreactors from EP 3 199 616 A1 and EP 3 246 393 A1 are also known from the prior art. The device in EP 3 246 393 A1 is characterized by reclosable vessel ports. In addition to different ports, the use of ATR elements made of the following materials; Ge, ZnSe, chalcogenides, glass was also described. However, this is the connection and not a permanently integrated single-use ATR element.

US 2010/0035337 A1 describes a bioreactor with a window. This window is distinguished by a photocatalytic coating. It is described that the window consists of an ATR crystal.

The publication by Glindkamp et al. (‘Sensors in disposable bioreactors’, Status and trends, Adv Biochem Eng Biotechnol. 2009) and Busse et al. (‘Sensors for disposable bioreactors’, Eng. Life Sci. 2017, 17, 940-952) reports on various sensors for bioreactors. The integration of ATR elements in disposable bioreactors is considered difficult since the costs for ATR elements are significantly too high.

Based on this, the object of this invention is to provide a bioreactor for single-use, in which the previous disadvantages occurring in the prior art are eliminated and allows a one-time contamination-free use with simultaneous continuous determination of measurement parameters.

This problem is solved by the bioreactor with the features of claim 1. Uses according to the invention are specified in claim 14. The further dependent claims show further developments.

According to the invention, a bioreactor for single-use is provided comprising a reactor housing and a silicon-comprising single-use ATR element, which is connected to the reactor housing and is connectable to an infrared spectrometer.

The infrared light is guided from the source via the single-use ATR element to the detector. Several parallel microprisms enable efficient coupling of the light. Due to the preferred use of parallel micro prisms the positioning accuracy of the optics is low.

Due to the tightly integrated single-use ATR element, parameters such as glucose, lactate and ammonium can now be measured continuously online. There is no need to take a sample from the bioreactor and contamination can be excluded.

The ATR element preferably comprises or consists of a single reflection element, the single reflection element preferably comprises or consisting of one or more microprisms.

In a further preferred embodiment, the ATR element comprises or consists of a multiple reflection element.

The ATR element is preferably an ATR infrared microscopy element.

The ATR element preferably consists of silicon or a silicon substrate with a coating. This coating can either be a thin layer, e.g., out of diamond, or represent a coating preventing the adhesion of cells.

The ATR element preferably has a round or angular shape. However, any other shape is also possible.

The ATR element is preferably connected air-tight to the side surfaces and/or the bottom of the bioreactor.

The infrared spectrometer preferably emits light in the wavelength range from 2 to 20 μm.

The reactor housing is preferably air-tight to prevent contamination. According to a preferred embodiment, the reactor housing is designed as a non-dimensionally stable container.

It is preferred that the reactor housing is made of a biocompatible polymer, in particular selected from the group consisting of polyether ether ketones (PEEK), polycarbonates (PC), polyether sulfones (PES), polysulfones (PS), polyvinyl chloride (PVC) and combinations thereof.

It is preferred that the bioreactor has sensors, in particular for determining the pH or the oxygen content in the bioreactor.

The bioreactor according to the invention, as described above, is used for the cultivation of microorganisms, cell cultures or for cell-free gene expression.

Based on the following figures, the subject matter of the invention is to be explained in more detail without wanting to restrict it to the specific form shown here.

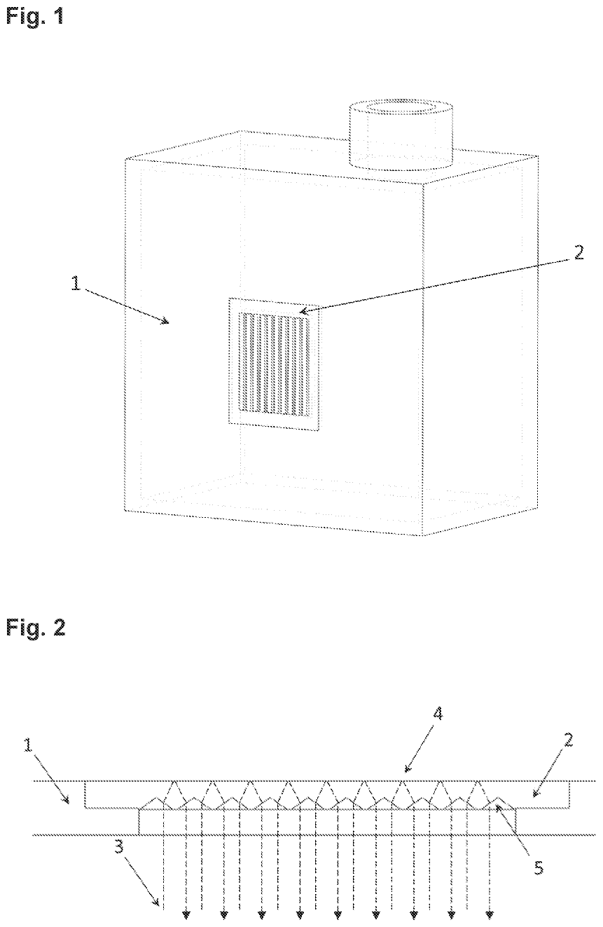

FIG. 1 shows a single-use bioreactor according to the invention.

FIG. 2 shows a detailed view of the single-use bioreactor according to the invention with an integrated ATR element.

FIG. 1 shows the single-use bioreactor (1) with the permanently integrated disposable ATR element (2). The single-use ATR element is integrated in the figure as an example on the side.

FIG. 2 shows the detailed view of the single-use bioreactor with a permanently integrated single-use ATR element and the light path within the stated ATR element. The infrared radiation (3) is coupled in and out through the microprisms (5). The interface between the sample in the single-use bioreactor (1) and the single-use ATR element (2), is where the attenuated total reflection occurs.

### LIST OF REFERENCE NUMBERS

Single-use bioreactor
Single-use ATR element
Infrared light
Attenuated total reflection
Microprisms

1-14. (canceled)

15. A bioreactor for single-use comprising a reactor housing and a silicon-containing ATR element, intended for single-use which is inseparably connected to the reactor housing and which is connectable to an infrared spectrometer.

16. The bioreactor according to claim 15, wherein the ATR element comprises a single reflection element.

17. The bioreactor according to claim 16, wherein the single reflection element comprises one or more microprisms.

18. The bioreactor according to claim 15, wherein the ATR element comprises a multiple reflection element.

19. The bioreactor according to claim 15, wherein the ATR element is an ATR infrared microscopy element.

20. The bioreactor according to claim 15, wherein the ATR element consists of silicon.

21. The bioreactor according to claim 15, wherein the ATR element consists of a silicon substrate with a coating.

22. The bioreactor according to claim 15, wherein the ATR element comprises a round or angular shape.

23. The bioreactor according to claim 15, wherein the ATR element is connected to the side surfaces and/or the bottom of the bioreactor in an air-tight manner.

24. The bioreactor according to claim 15, wherein the infrared spectrometer emits light in the wavelength range from 2 to 20 μm.

25. The bioreactor according to claim 15, wherein the reactor housing is air-tight to prevent contamination.

26. The bioreactor according to claim 15, wherein the reactor housing is designed as a non-dimensionally stable container.

27. The bioreactor according to claim 15, wherein the reactor housing is made from a polymer.

28. The bioreactor according to claim 27, wherein the polymer is selected from the group consisting of polyether ether ketones (PEEK), polycarbonates (PC), polyether sulfones (PES), polysulfones (PS), polyvinyl chlorides (PVC), and combinations thereof.

29. The bioreactor according to claim 15, wherein the bioreactor comprises a sensor or sensors.

30. The bioreactor according to claim 29, wherein the sensor or sensors is/are for determining the pH or oxygen content in the bioreactor.

31. The bioreactor according to claim 29, wherein the sensors are connected airtight to the reactor housing.

32. A method of cultivating of microorganisms or cell cultures or causing cell-free gene expression, the method comprising utilizing the bioreactor according to claim 15 and cultivating of microorganisms or cell cultures or causing cell-free gene expression.
